(19) United States
(12) Patent Application Publication
(10) Pub. No.: US 2024/0181115 A1
(43) Pub. Date: Jun. 6, 2024

(54) MODELED UVC RAY EMITTER

(71) Applicant: ALUVIA-ENGENHARIA E INVESTIGACAO, LDA., PORTO (PT)

(72) Inventors: Carlos Manuel ROCHA AMARO NEVES, VILA NOVA DE GAIA (PT); Antonio Paulo DE SOUSA CALIX, ESPINHO (PT)

(21) Appl. No.: 18/552,803

(22) PCT Filed: Mar. 29, 2022
(86) PCT No.: PCT/IB2022/052896

§ 371 Date: Sep. 27, 2023

(30) Foreign Application Priority Data
Mar. 30, 2021 (PT) 117153

(51) Int. Cl. A61L 9/20; A61L 2/10

(52) U.S. Cl. CPC A61L 9/20; A61L 2/10; A61L 2202/14; A61L 2209/11

(57) ABSTRACT

A modeled UVC ray emitting apparatus (100) that automatically and sequentially irradiates different wavelengths, in the entire range of the UVC wavelength spectrum, namely between 200 nm and 280 nm, guaranteeing a significant increase in the effectiveness of the sterilisation of the areas in which it is placed. The apparatus is composed of a stationary UVC ray emitting source (1) which promotes the variation of the wavelength through a set of tubes circumscribing the emitting source, as well as the fluid circulating inside the tubes.

### TECHNICAL FIELD

The present invention describes a modeled UVC ray emitting apparatus.

### BACKGROUND ART

Ultraviolet (UV) radiation is a well-known disinfectant for air, water and non-porous surfaces that has been effectively used for decades to reduce the spread of bacteria, organisms, viruses, fungi, etc.

The ability of this radiation in destroying this type of organisms is mostly within the range of the wavelength spectrum between 200 nm and 280 nm, that is, in the UVC range. For this reason, UVC lamps, often used for this purpose, are designated “germicidal” lamps.

However, and under the same conditions of intensity, exposure time and proximity to the emitting source, studies have shown that each pathogen agent responds differently and uniquely to the different wavelengths of said rays.

The equipment developed to date resorts to the use of a multiplicity of UV ray emitting sources with fixed wavelengths to guarantee a minimally effective result.

In the present approach, the aim is to overcome this state of the art limitation, guaranteeing the coverage of the entire UVC ray spectrum, focusing more effectively on the vulnerabilities of the type of agent to be targeted.

### SUMMARY

The present application describes a modeled UVC ray emitting apparatus, characterized in that it comprises a support base; at least one stationary UVC ray emitting source adapted to an upper face of the support base in a central position; and a flow circuit adapted on the upper face of the support base, completely circumscribing the at least one stationary UVC ray emitting source in a central position; wherein the flow circuit models the wavelength emitted by the at least one stationary UVC ray emitting source.

In a proposed embodiment, the modeled UVC ray emitting apparatus comprises a thermal drive with flow variator.

In yet another embodiment, the flow circuit comprises a set of tubes made of a material with non-interfering characteristics in the radiation of the UVC spectrum.

In yet another embodiment, the set of tubes made of a material with non-interfering characteristics in the radiation of the UVC spectrum comprises within it the circulation of a fluid.

In yet another embodiment, the fluid is subject to temperature variation and pressure variation.

In yet another embodiment, the thermal drive with flow variator is adapted to promote the circulation of fluid within the flow circuit.

In yet another embodiment, the thermal drive with flow variator is adapted to promote the temperature variation of the fluid within the flow circuit.

In yet another embodiment, the thermal drive with flow variator comprises a pressure pump.

In yet another embodiment, the thermal drive with flow variator is adapted to promote the pressure variation of the fluid within the flow circuit.

In yet another embodiment, the temperature variation of the fluid within the flow circuit comprises values between 2° C. and 22° C., and preferably between 4° C. and 20° C.

In yet another embodiment, the fluid pressure variation within the flow circuit comprises values between 0.515568 kg/cm² and 1.000000 kg/cm².

In yet another embodiment, the flow circuit comprises a confluence ring adapted to ensure confluence of fluids within the flow circuit.

In yet another embodiment, the flow circuit comprises an expansion box, located in the confluence ring, responsible for ensuring the balance of the internal pressure in the circuit caused by the fluid circulating inside thereof, ensuring the accommodation of the fluid in expansion and compression moments.

In yet another embodiment, the flow circuit models the wavelength emitted by the at least one stationary UVC ray emitting source in a range between 200 nm and 280 nm.

The present application further describes the method of operation of the modeled UVC ray emitting apparatus according to the foregoing description comprising the steps of: 1. initializing the flow circuit; 2. After two minutes of the initialization process, the temperature of the fluid running through the flow circuit is raised and stabilized at 20° C.; 3. the emitting source lamp is activated; 4. After five minutes of emitting source lamp activation, the process of gradual cooling of the fluid running through the flow circuit begins over a period of five minutes until the temperature of the fluid running through the flow circuit stabilizes at 4° C.; 5. Once the temperature of the fluid running through the flow circuit has stabilized at 4° C., the emitting source lamp is deactivated so that the pressure of the mercury vapor inside drops, the operating cycle returning to the step described in 2.

### BRIEF DESCRIPTION

The present invention concerns an apparatus that automatically and sequentially irradiates different wavelengths, in the entire range of the UVC ray wavelength spectrum, namely between 200 nm and 280 nm.

The developed apparatus comprises the use of a fixed wavelength emitting source, allowing to guarantee the entire range of said spectrum between 200 nm and 280 nm through a modeling system of said UVC ray wavelength.

By using this apparatus, it is possible to guarantee the coverage of a wider spectrum, resulting, therefore, in a significant increase in the sterilization efficiency of the spaces. Thus, and with the approach of the developed system, each bacteriological and/or viral agent will be irradiated within the wavelength range for which it presents greater vulnerability, thus making it possible to increase the effectiveness of the elimination of these agents through a reduction of the exposure period required in about a third of the time when compared to existing systems, thus also increasing the universe of possible applications.

### BRIEF DESCRIPTION OF THE FIGURES

For an easier understanding of the present application, figures are herein attached, which represent embodiments which however are not intended to limit the art herein disclosed.

FIG. 1 shows a view of the proposed apparatus. Reference numbers relate to:
100. Modeled UVC ray emitting apparatus;
1. stationary UVC ray source;
2. flow circuit;
3. thermal variator with flow control;
4. support base;
5. locomotion system;
6. confluence ring.

FIG. 2 shows the electromagnetic spectrum and the spectral curve of cell inactivation between UVC and UVB ray spectra, also referencing the wavelength of the stationary UVC ray emitting source (1) at 254 nm.

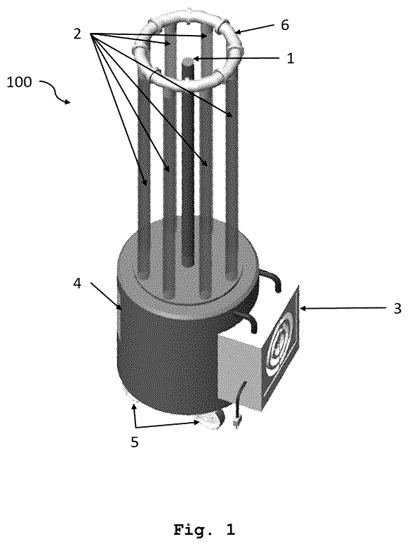

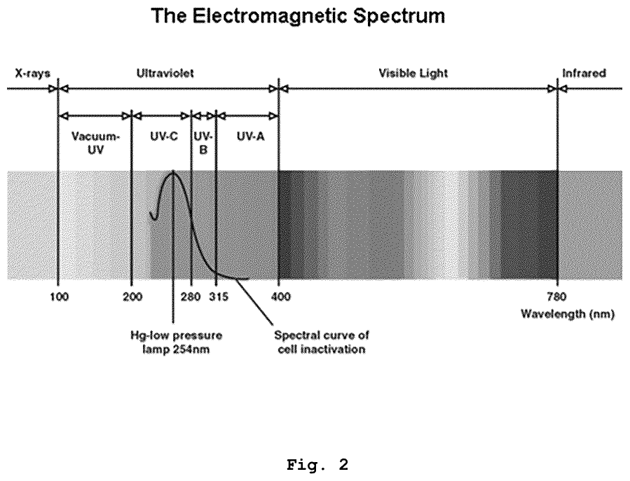

### DESCRIPTION OF THE EMBODIMENTS

Referring to the figures, some embodiments are now described in more detail, which are not intended, however, to limit the scope of the present application.

The starting point for the development of the apparatus (100) is based on the knowledge that electromagnetic waves always move at a speed of 299.792 km/sec in vacuum, and when these waves cross different matter, their propagation speed is reduced. This phenomenon is related to the fact that light is dispersed among molecules making up the different media and does not affect photon speed. Thus, and for a given light frequency, the wavelength is proportional to wave speed,

c=ν×λ,

wherein c represents speed, ν represents frequency and λ the wavelength. Therefore, for the case where a reduction in speed c is observed, the wavelength λ will also decrease, keeping the frequency ν unchanged. Thus, the denser the medium to be traversed, the shorter the wavelength, and vice versa.

Thus, and based on this principle, the present application describes a modeled UVC ray emitting apparatus (100) comprising a stationary UVC ray emitting source (1), installed in a central position with respect to a flow circuit (2), or alternator system. The flow circuit (2) laterally circumscribes the stationary UVC source (1) and is responsible for modeling the wavelength emitted by the source (1), thus guaranteeing UVC ray emission covering the entire range of values between 200 nm and 280 nm.

The support base (4) of the modeled UVC ray emitting apparatus (100) comprises the use of a housing base for electrical and electronic circuits, being adapted to guarantee the safety and operation of the various modules of the emitter (100), and a cooling circuit arranged on the outside.

For one of the proposed embodiments, the UVC ray emitting source (1) of the apparatus (100) comprises a mercury vapor lamp, which at the beginning of its activity (start-up) emits a wavelength at 288 nm, only reaching the peak of stable performance with a wavelength at 254 nm after approximately five minutes, at which time there is sufficient vapor inside the lamp capable of generating the pressure necessary for the reaction. During the five heating minutes of the emitting source (1) lamp, and since during this heating the emitted rays cover wavelengths between 288 nm and 254 nm, said heating promotes by itself, the germicidal effect within such range. Once the stabilized/stationary operating point is reached, the remaining modeling of the rays emitted between 254 nm and 200 nm is therefore guaranteed by refraction through incidence thereof in the flow circuit (2).

The flow circuit (2), in one of the non-limiting proposed embodiments, comprises the use of a closed circuit of tubes, which in the perspective of increasing the versatility of the device (100), may comprise the use of multiple independent circuits. The flow circuit (2) comprises several transparent and/or translucent tubes arranged vertically, eight in one of the preferred non-limiting embodiments, which are interconnected by means of a closed confluence ring (6) which may be made of PVC or other material duly appropriate for the purpose. The vertical tubes of the flow circuit (2) have at least the same length as the stationary UVC ray emitting source (1) and are structurally constructed using a pure quartz composition so as not to interfere with the UVC spectrum emitted by the stationary source (1). The tubes of the flow circuit (2) comprise the use of a material with non-interfering characteristics in the radiation of the UVC spectrum, in one of the proposed embodiments in carbon-free pure quartz and approximately 2 mm thick, within which a fluid circulates, which, in the proposed embodiment, when subjected to thermal variations in a range between 2° C. and 22° C., ideally between 4° C. and 20° C., undergoes changes in density, respectively between 1000 g/cm³ and 0.998232 g/cm³, and pressure, respectively between 0.515568 kg/cm² and 1.000000 kg/cm². The circulating fluid may, in one of the several possible embodiments, comprise deionized and/or desalinated water to reduce the levels of sodium crystals, but alternatively other types of solutions may be used, namely electrically controlled variable density gel, gases, or others technically suitable for the purpose. The flow circuit (2) further comprises an expansion box, located in the confluence ring (6), responsible for ensuring the balance of the internal pressure in the circuit caused by the fluid circulating inside it, ensuring the accommodation of the expanded fluid at times of temperature rise.

The thermal variator with flow control (3) shall be responsible for the thermal control of the fluid inside the flow circuit tubes (2), as well as for controlling the flow and its circulation within. The thermal variator (3) comprises the use of a radiator to carry out the fluid cooling process, and a resistor to guarantee the heating thereof, a thermostat being used in both procedures to guarantee the accuracy of the temperature reading. The thermal variator (3) is pre-programmable to allow the programming of operating cycles according to the need, making it possible not only to define the temperature ranges, and corresponding range of UVC incidence, but also their action period. This programming allows adjusting the operation of the apparatus for specific functions, namely the elimination of specific viruses by exposure to particular wavelengths. The thermal variator with flow control (3) also comprises a pressure pump that is responsible for controlling the pressure of the fluid present within the flow circuit (2), guaranteeing, together with the expansion box, the integrity and structural life span of the flow circuit tubes (2). Changing temperature, density and pressure of the fluid present inside the flow circuit (2) will promote the change in the wavelength of the rays emitted by the emitting source (1) when they cross the tubes. The forced circulation of the fluid inside the circuit tubes (2), as well as the control of temperature variations thereof, are ensured by a flow thermo-control device, designated thermal variator with flow control (3). Stabilizing the temperature at certain levels allows different values to be obtained along the aforementioned range, obtaining, for example, wavelengths between 235 nm and 240 nm. The versatility in obtaining different wavelengths makes it possible to define more clearly which virological and/or bacterial agents are to be eliminated. Thus, and with the possibility of varying the incident wavelengths, it is possible to optimize and improve the performance of the devices created for this purpose, since this variation of the emitted wavelength will allow to reduce the overdose of wavelengths, allowing a linear variation throughout the entire spectrum covered, and the reduction of exposure times in the surrounding application areas.

In a non-limiting and exemplifying way, the fluid present inside the flow circuit (2), at a temperature of 4° C., promotes a deviation in the wavelength of the UVC emitted by the emitting source (1) to the 201 nm range. For this temperature range, and in order to achieve the desired wavelength change, and using the pressure pump of the thermal variator with flow control (3), an approximate pressure of 0.515568 kg/cm² is imposed on the fluid. Analogously, to obtain a deviation in the wavelength of the UVC emitted by the emitting source (1) to the 215 nm range, the corresponding temperature of the fluid shall be approximately 7° C. and a pressure of 0.534748 kg/cm². In order to obtain a deviation in the wavelength of the UVC emitted by the emitting source (1) for the 240 nm range, the corresponding temperature of the fluid shall be approximately 12° C. and a pressure of 0.645718 kg/cm².

The correlation between temperature, density and pressure of the fluid, associated with the variation and modification of the wavelength, is susceptible of non-limiting individual regulation for each type of application of the emitting apparatus (100). It should be noted that by way of example and for the particular case described, the proposed density for the fluid is 1.0000 g/cm³ or presents decimal and/or centesimal values very close thereto, presenting very slight variations.

The calibration and programming of the apparatus (100) regarding wavelengths emitted between 200 nm and 280 nm, can be guaranteed using a spectrometer, ensuring that a ten-minute operating cycle covers the entire aforementioned UVC spectrum.

The typology used also makes it possible to optimize and considerably lower the energy consumption of the apparatus (100), with its overall consumption being approximately 500 watts. In terms of dimensions, in one of the proposed embodiments, the modeled UVC ray emitting apparatus (100) will have a height of about 1.5 m and a width of 0.5 m. The stationary UVC ray emitting source (1), in one of the proposed non-limiting embodiments, comprises the use of a UVC ray emitting lamp with 350 W power, with approximate dimensions of 1.20 m×20 mm.

The method of operation of the modeled UVC ray emitting apparatus (100), in one of the proposed embodiments, comprises the steps of: 1. initializing the flow circuit (2); 2. after two minutes of the initialization process, the temperature of the fluid running through the flow circuit (2) is raised and stabilized at 20° C.; 3. the emitting source lamp (1) is activated; 4. After five minutes of emitting source lamp (1) activation, the process of gradual cooling of the fluid running through the flow circuit (2) begins over a period of five minutes until the temperature of the fluid running through the flow circuit (2) stabilizes at 4° C.; 5. Once the temperature of the fluid running through the flow circuit (2) has stabilized at 4° C., the emitting source lamp (1) is deactivated so that the pressure of the mercury vapor inside drops, the operating cycle returning to the step described in 2.

The present description is of course in no way restricted to the embodiments presented herein and a person of ordinary skill in the art may provide many possibilities of modifying it without departing from the general idea as defined in the claims. The preferred embodiments described above are obviously combinable with each other. The following claims further define preferred embodiments.

1. A modeled UVC ray emitting apparatus (100), comprising:
a support base (4);
at least one stationary UVC ray emitting source (1) adapted to an upper face of the support base (4) in a central position; and
a flow circuit (2) adapted on the upper face of the support base (4) completely circumscribing the at least one stationary UVC ray emitting source (1) in a central position;
wherein flow circuit (2) models the wavelength emitted by the at least one stationary UVC ray emitting source (1).

2. The modeled UVC ray emitting apparatus (100) according to claim 1, comprising a thermal drive with flow variator (3).

3. The modeled UVC ray emitting apparatus (100) according to claim 1, wherein the flow circuit (2) comprises a set of tubes made of a material with non-interfering characteristics in the radiation of the UVC spectrum.

4. The modeled UVC ray emitting apparatus (100) according to claim 1, wherein the set of tubes, made of a material with non-interfering characteristics in the radiation of the UVC spectrum, comprises within it the circulation of a fluid.

5. The modeled UVC ray emitting apparatus (100) according to claim 4, wherein the fluid is subject to temperature variation and pressure variation.

6. The modeled UVC ray emitting apparatus (100) according to claim 2, wherein the thermal drive with flow variator (3) is adapted to promote the circulation of fluid within the flow circuit (2).

7. The modeled UVC ray emitting apparatus (100) according to claim 2, wherein the thermal drive with flow variator (3) is adapted to promote the temperature variation of the fluid within the flow circuit (2).

8. The modeled UVC ray emitting apparatus (100) according to claim 2, wherein the thermal drive with flow variator (3) comprises a pressure pump.

9. The modeled UVC ray emitting apparatus (100) according to claim 2, wherein the thermal drive with flow variator (3) is adapted to promote the pressure variation of the fluid within the flow circuit (2).

10. The modeled UVC ray emitting apparatus (100) according to claim 5, wherein the temperature variation of the fluid within the flow circuit (2) comprises values between 2° C. and 22° C.

11. The modeled UVC ray emitting apparatus (100) according to claim 5, wherein the fluid pressure variation within the flow circuit (2) comprises values between 0.515568 kg/cm² and 1.000000 kg/cm².

12. The modeled UVC ray emitting apparatus (100) according to claim 1, wherein the flow circuit (2) comprises a confluence ring (6) adapted to ensure the confluence of fluids within the flow circuit (2).

13. The modeled UVC ray emitting apparatus (100) according to claim 12, wherein the flow circuit (2) comprises an expansion box, located in the confluence ring (6), responsible for ensuring the balance of internal pressure in the circuit caused by the fluid circulating inside thereof, ensuring the accommodation of the fluid in expansion and compression moments.

14. The modeled UVC ray emitting apparatus (100) according to claim 1, wherein the flow circuit (2) models the wavelength emitted by the at least one stationary UVC ray emitting source (1) in a range between 200 nm and 280 nm.

15. A method of operation of the modeled UVC ray emitting apparatus (100) according to claim 1 comprising the steps of:
(a) initializing the flow circuit (2);
(b) after two minutes of the initialization process, the temperature of the fluid running through the flow circuit (2) is raised and stabilized at 20° C.;
(c) the emitting source lamp (1) is activated;
(d) after five minutes of emitting source lamp (1) activation, the process of gradual cooling of the fluid running through the flow circuit (2) begins over a period of five minutes until the temperature of the fluid running through the flow circuit (2) stabilizes at 4° C.;
(e) once the temperature of the fluid running through the flow circuit (2) has stabilized at 4° C., the emitting source lamp (1) is deactivated so that the pressure of the mercury vapor inside drops, the operating cycle returning to the step described in step (b).

16. The modeled UVC ray emitting apparatus (100) according to claim 10, wherein the temperature variation of the fluid within the flow circuit (2) comprises values between 4° C. and 20° C.
